(19) United States
(12) Patent Application Publication
(10) Pub. No.: US 2016/0338603 A1
(43) Pub. Date: Nov. 24, 2016

(54) SIGNAL PROCESSING DEVICE, SIGNAL PROCESSING METHOD, AND COMPUTER-READABLE RECORDING MEDIUM

(71) Applicant: FUJITSU LIMITED, Kawasaki-shi (JP)

(72) Inventors: Yasuyuki NAKATA, Zama (JP); Akihiro INOMATA, Atsugi (JP)

(73) Assignee: FUJITSU LIMITED, Kawasaki-shi (JP)

(21) Appl. No.: 15/228,862

(22) Filed: Aug. 4, 2016

Related U.S. Application Data
(63) Continuation of application No. PCT/JP2014/053380, filed on Feb. 13, 2014.

(51) Int. Cl. A61B 5/024; G06K 9/62; G06T 7/00; G06K 9/46; A61B 5/0402; A61B 5/00

(52) U.S. Cl. CPC A61B 5/02416; A61B 5/0402; A61B 5/7475; A61B 5/742; A61B 5/0022; A61B 5/7203; A61B 5/725; G06T 7/0012; G06K 9/4604; G06K 9/6282; A61B 2562/0233; G06T 2207/30101

(57) ABSTRACT

A pulse wave detection device acquires an image. The pulse wave detection device extracts a living body region included in the image. The pulse wave detection device generates a signal from time series data of a pixel value included in a partial image of the image corresponding to the living body region. The pulse wave detection device calculates a variation index for evaluating a degree of disturbance of a pulse wave included in the signal. The pulse wave detection device controls whether to output the signal by using the variation index.

### CROSS-REFERENCE TO RELATED APPLICATION

This application is a continuation of International Application No. PCT/JP2014/053380, filed on Feb. 13, 2014, the entire contents of which are incorporated herein by reference.

### FIELD

The embodiments discussed herein are related to a signal processing device, a signal processing method, and a signal processing program.

### BACKGROUND

As an example of a technique for detecting fluctuations in volume of blood, what is called a pulse wave, developed are a biological state detection device and a pulsimeter described below.

Of these, the biological state detection device detects a signal of reflected light related to green light and infrared light by causing a green light emitting diode (LED) and an infrared LED arranged in a pulse wave sensor fitted to an arm and the like of a human body to emit light alternately. The biological state detection device then performs frequency analysis on a detection signal obtained for each of the green light and the infrared light. Thereafter, the biological state detection device extracts a frequency that is present in a frequency analysis result of the green light and not present in a frequency analysis result of the infrared light, and converts an extracted peak frequency into a pulse rate.

The pulsimeter evaluates a target pulse width Px based on a pulse width evaluation range at a plurality of stages, and performs a processing operation such as updating a reference pulse width P, not updating the reference pulse width P, complementing pulse data for one beat, and discarding data of the target pulse width Px based on an evaluation result. Due to this, a signal recognized as a regular signal of a pulse width of a pulse is extracted to be transmitted to a rear stage.

Patent document 1: Japanese Laid-open Patent Publication No. 2012-170703

Patent document 2: Japanese Laid-open Patent Publication No. 05-184548

Patent document 3: Japanese Laid-open Patent Publication No. 2004-261390

Patent document 4: Japanese Laid-open Patent Publication No. 2004-261366

Patent document 5: Japanese Laid-open Patent Publication No. 2002-102185

However, with the technique described above, output control is difficult to be appropriately performed on a detection result of the pulse wave in some cases.

That is, with the biological state detection device described above, the pulse rate continues to be calculated even when a noise removal function does not work because noise at a level at which a component corresponding to the pulse wave is difficult to be extracted is superimposed on the signal, so that an abnormal pulse rate may be displayed. With the pulsimeter, when noise having the target pulse width Px similar to the reference pulse width P is superimposed on the signal, the signal is not discarded and directly transmitted to a rear stage in some cases.

### SUMMARY

According to an aspect of an embodiment, a signal processing device includes a processor that executes a process. The process includes: acquiring an image; extracting a living body region included in the image; first generating a signal from time series data of a pixel value included in a partial image of the image corresponding to the living body region; calculating a variation index for evaluating a degree of disturbance of a pulse wave included in the signal; and controlling whether to output the signal by using the variation index.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a block diagram illustrating a functional configuration of a pulse wave detection device according to a first embodiment;

FIG. 2 is a diagram illustrating an example of a spectrum of each of a G signal and an R signal;

FIG. 3 is a diagram illustrating an example of the spectrum of each signal of a G component and an R component multiplied by a correction coefficient k;

FIG. 4 is a diagram illustrating an example of the spectrum after an arithmetic operation;

FIG. 5 is a block diagram illustrating a functional configuration of a generation unit illustrated in FIG. 1;

FIG. 6 is a diagram illustrating an example of the spectrum of a pulse wave signal;

FIG. 7 is a diagram illustrating an example of the spectrum of the pulse wave signal;

FIG. 8 is a diagram illustrating an example of a waveform of the pulse wave signal;

FIG. 9 is a diagram illustrating an example of the waveform of the pulse wave signal;

FIG. 10 is a diagram illustrating an example of the waveform of the pulse wave signal;

FIG. 11 is a flowchart illustrating a signal processing procedure according to the first embodiment;

FIG. 12 is a block diagram illustrating a functional configuration of a determination model generation device according to a second embodiment;

FIG. 13 is a diagram illustrating an example of a determination model;

FIG. 14 is a diagram illustrating an example of a classification result based on a fluctuation in a difference between adjacent extreme values and a peak ratio;

FIG. 15 is a diagram illustrating an example of a classification result based on an area of spectral distribution and the peak ratio;

FIG. 16 is a flowchart illustrating a setting processing procedure of the determination model according to the second embodiment; and

FIG. 17 is a diagram for explaining an example of a computer that executes a signal processing program according to the first embodiment to a third embodiment.

### DESCRIPTION OF EMBODIMENTS

Preferred embodiments will be explained with reference to accompanying drawings. The embodiments do not limit the disclosed technique. The embodiments can be appropriately combined in a range in which contradiction is not caused in processing content.

### [a] First Embodiment

### Configuration of Pulse Wave Detection Device

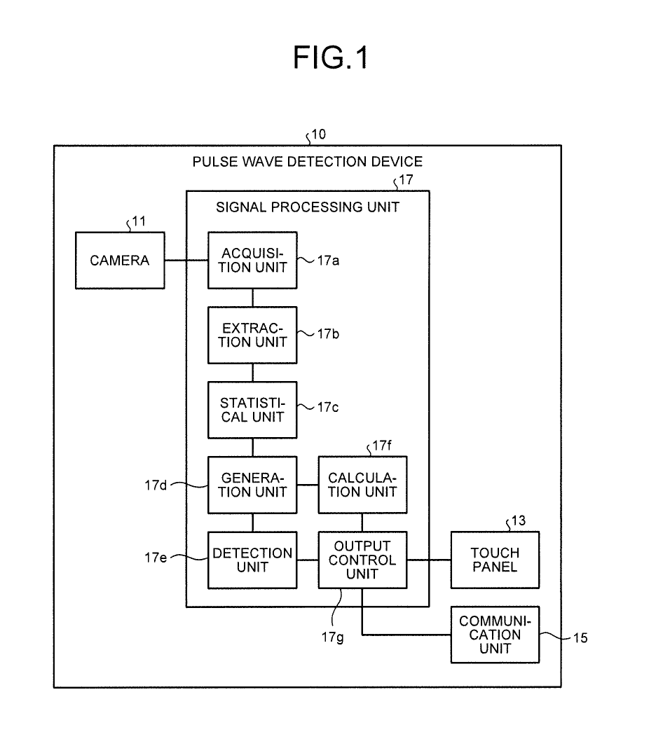

First, the following describes a functional configuration of a pulse wave detection device according to a first embodiment. FIG. 1 is a block diagram illustrating a functional configuration of the pulse wave detection device according to the first embodiment. A pulse wave detection device 10 illustrated in FIG. 1 performs pulse wave detection processing of detecting a pulse wave of a subject, that is, fluctuations in volume of blood caused by heartbeat by using an image obtained by photographing a living body of the subject without bringing a measuring instrument into contact with the subject under typical environmental light such as sunlight or indoor light. As part of such pulse wave detection processing, the pulse wave detection device 10 determines quality of a pulse wave signal generated from the image obtained by photographing the living body, and performs signal processing for preventing a poor pulse wave signal from being output.

According to one aspect, the pulse wave detection device 10 can be implemented by installing, in a desired computer, a signal processing program in which the signal processing is provided as package software or online software. For example, the signal processing program is installed not only in a mobile object communication terminal such as a smartphone, a mobile phone, and a personal handyphone system (PHS) but also in a portable terminal device including a digital camera, a tablet terminal, and a slate terminal having no capability of being connected to a mobile object communication network. This configuration can cause the portable terminal device to function as the pulse wave detection device 10. As an implementation example of the pulse wave detection device 10, the portable terminal device is exemplified herein. Alternatively, the signal processing program can be installed in a stand-alone type terminal device including a personal computer.

As illustrated in FIG. 1, the pulse wave detection device 10 includes a camera 11, a touch panel 13, a communication unit 15, and a signal processing unit 17.

The pulse wave detection device 10 illustrated in FIG. 1 may include various functional units included in a known computer in addition to the functional units illustrated in FIG. 1. For example, when the pulse wave detection device 10 is implemented as a tablet terminal or a slate terminal, the pulse wave detection device 10 may further include a motion sensor such as an acceleration sensor and a gyro sensor. When the pulse wave detection device 10 is implemented as a mobile object communication terminal, the pulse wave detection device 10 may further include a functional unit such as an antenna and a global positioning system (GPS) receiver. As an example, FIG. 1 exemplifies the functional units in a case in which the pulse wave detection device 10 is implemented as a portable terminal device. However, it goes without saying that the pulse wave detection device 10 can be implemented as a stand-alone terminal. For example, when the pulse wave detection device 10 is implemented as a stand-alone terminal, the pulse wave detection device 10 may include an input/output device such as a keyboard, a mouse, and a display.

The camera 11 is an imaging device including an imaging element such as a charge coupled device (CCD) and a complementary metal oxide semiconductor (CMOS) mounted therein. For example, three or more types of light receiving elements such as R (red), G (green), and B (blue) can be mounted in the camera 11. As an implementation example of the camera 11, a digital camera or a Web camera may be connected via an external terminal. As another implementation example, when a camera is mounted therein before shipment like an in-camera or an out-camera, the camera can be used. Although a case in which the pulse wave detection device 10 includes the camera 11 is exemplified herein, the pulse wave detection device 10 does not necessarily include the camera 11 when an image can be acquired via a network or a storage device.

For example, the camera 11 can take a rectangular image of 320 pixels in a horizontal direction×240 pixels in a vertical direction. For example, in a case of a gray scale, each pixel is represented by a gradation value of brightness (luminance). For example, the gradation value of luminance (L) of a pixel at coordinates (i, j) indicated by integral numbers i and j is represented by a digital value L(i, j) of 8-bit. In a case of a color image, each pixel is represented by gradation values of an R component, a G component, and a B component. For example, gradation values of R, G, and B of the pixel at the coordinates (i, j) indicated by the integral numbers i and j are represented by digital values R(i, j), G(i, j), and B(i, j). A combination of RGB or another color system obtained by converting RGB values (an HSV color system or a YUV color system) may be used.

The touch panel 13 is a device that can perform display and input. According to one aspect, the touch panel 13 displays an image output by a signal processing program executed on the pulse wave detection device 10, an operating system (OS), and an application program. According to another aspect, the touch panel 13 receives a touch operation such as tapping, flicking, sweeping, pinch-in, and pinch-out performed on a screen. In this case, the touch panel 13 is exemplified as an input device for inputting an instruction to the pulse wave detection device 10. However, the present invention is not limited thereto. A physical key and the like for implementing a complementary input with respect to the touch panel 13 may be further provided.

When the signal processing program described above is started, a photographing operation of an image can be guided so that an image of the subject from which a pulse wave is easily detected is taken by the camera 11 through image display performed by the touch panel 13 or a voice output from a speaker (not illustrated). For example, when being started via the touch panel 13, the signal processing program starts the camera 11. Thereafter, the camera 11 starts to photograph the subject accommodated in a photographing range of the camera 11. To photograph the image in which a face of the subject is reflected, the signal processing program can cause the touch panel 13 to perform display aiming at a target position at which a nose of the subject is reflected while displaying the image photographed by the camera 11 on the touch panel 13. Due to this, the camera 11 can photograph the image in which, among face parts of the subject such as an eye, an ear, a nose, and a mouth, the nose of the subject is accommodated in the center portion of the photographing range. The signal processing program outputs the image of the face of the subject photographed by the camera 11 to the signal processing unit 17. The guidance described above is not necessarily performed. The face of the subject can be photographed during a time in which the subject views a screen displayed on the touch panel 13, for example, an image or a moving image output from an operating system or an application program. Accordingly, photographing can be performed in a background without causing the subject to be aware of photographing.

The communication unit 15 is an interface that performs communication control between itself and another device (not illustrated). As an aspect of the communication unit 15, a network interface card, what is called an NIC can be employed. For example, the communication unit 15 transmits a pulse wave output through the signal processing, for example, a pulse rate or a pulse waveform to a server device (not illustrated), and receives a diagnostic result and the like diagnosed by the server device based on the pulse rate and the pulse waveform.

The signal processing unit 17 is a processing unit that performs the signal processing described above. The signal processing unit 17 includes, as illustrated in FIG. 1, an acquisition unit 17a, an extraction unit 17b, a statistical unit 17c, a generation unit 17d, a detection unit 17e, a calculation unit 17f, and an output control unit 17g.

Of these, the acquisition unit 17a is a processing unit that acquires the image. According to one aspect, the acquisition unit 17a can acquire the image taken by the camera 11. According to another aspect, the acquisition unit 17a can acquire the image from an auxiliary storage device such as a hard disk or an optical disc that accumulates images, or a removable medium such as a memory card or a universal serial bus (USB) memory. According to yet another aspect, the acquisition unit 17a can acquire the image by receiving the image from an external device via a network. Exemplified is a case in which the acquisition unit 17a performs processing by using an image such as two-dimensional bit map data or vector data obtained from an output by an imaging element such as a CCD or a CMOS. Alternatively, a signal output from one detector may be directly acquired to perform processing at a rear stage.

The extraction unit 17b is a processing unit that extracts a living body region from the image. According to one aspect, the extraction unit 17b extracts a face region based on a predetermined face part from the image acquired by the acquisition unit 17a. For example, by executing face recognition such as template matching on the image, the extraction unit 17b detects, from among organs of a face such as an eye, an ear, a nose, and a mouth of the subject, what is called face parts, a specific face part, that is, the nose of the subject. Subsequently, the extraction unit 17b extracts a face region included in a predetermined range centered on the nose of the subject. Due to this, a partial image of the face region including a face center part including the nose of the subject and part of cheeks positioned around the nose is extracted as an overall image used for detecting a pulse wave. Thereafter, the extraction unit 17b outputs the partial image corresponding to the face region extracted from the image to the statistical unit 17c. As an example of the living body region, the face region is extracted herein. However, an extracted part is not necessarily the face. Any part may be used so long as skin is reflected therein.

The statistical unit 17c is a processing unit that performs predetermined statistical processing on a pixel value of each pixel of the partial image corresponding to the living body region. According to one aspect, the statistical unit 17c averages luminance values of pixels of the partial image corresponding to the face region for each wavelength component of RGB. In addition to an average value, a median or a mode may be calculated. In addition to an arithmetic mean, optional averaging processing such as weighted average or a moving average can be performed. Accordingly, the average value of the luminance of the pixels of the partial image corresponding to the face region is calculated for each of RGB components as a representative value representing the face region.

The generation unit 17d is a processing unit that generates a signal of a frequency component corresponding to the pulse wave from a signal of the representative value for each wavelength component of the partial image corresponding to the living body region. According to one aspect, by performing signal generation processing described below, the generation unit 17d generates, from a signal of the representative value for each wavelength component of the partial image corresponding to the face region, a pulse wave signal in which components in a specific frequency band are canceled with each other, the specific frequency band other than a pulse wave frequency band that may be employed by the pulse wave among a plurality of wavelength components. Hereinafter, the signal in which noise is canceled through signal generation processing may be referred to as a “pulse wave signal”. For example, the generation unit 17d detects the pulse wave signal by using time series data of representative values of two wavelength components including the R component and the G component having different light absorption characteristics of blood among three wavelength components, that is, the R component, the G component, and the B component.

Specifically, a capillary passes through a face surface, and when a blood flow flowing in a blood vessel is changed due to a heartbeat, an amount of light absorbed by the blood flow is also changed depending on the heartbeat. Accordingly, luminance obtained with reflection from the face is changed in accordance with the heartbeat. Although a change amount of the luminance is small, a pulse wave component is included in the time series data of luminance when average luminance of the entire face region is obtained. However, the luminance is also changed due to body motion and the like in addition to the pulse wave, which becomes a noise component in pulse wave detection, what is called a body motion artifact. Thus, the pulse wave is detected using two or more types of wavelengths having different light absorption characteristics of blood, for example, the G component having a high light absorption characteristic (about 525 nm) and the R component having a low light absorption characteristic (about 700 nm). The heartbeat is within a range from 0.5 Hz to 4 Hz, that is, from 30 bpm to 240 bpm for one minute, so that other components can be regarded as noise components. Assuming that there is no wavelength characteristic in noise, or the wavelength characteristic is extremely small if any, components other than 0.5 Hz to 4 Hz are assumed to be the same between the G signal and the R signal. However, a size of the component is different depending on a difference in sensitivity of the camera. Thus, when the difference in sensitivity of the components other than 0.5 Hz to 4 Hz is corrected and the R component is subtracted from the G component, the noise component is removed and only the pulse wave component can be extracted.

For example, the G component and the R component can be represented by the following expressions (1) and (2). In the following expression (1), “Gs” indicates the pulse wave component of the G signal, and “Gn” indicates the noise component of the G signal. In the following expression (2), “Rs” indicates the pulse wave component of the R signal, and “Rn” indicates the noise component of the R signal. There is a difference in sensitivity for the noise component between the G component and the R component, so that a correction coefficient k for the difference in sensitivity is represented by the following expression (3).

Ga=Gs+Gn (1)

Ra=Rs+Rn (2)

k=Gn/Rn (3)

When the difference in sensitivity is corrected and the R component is subtracted from the G component, a pulse wave component S is represented by the following expression (4). When the expression (4) is converted into an expression represented with Gs, Gn, Rs, and Rn by using the expressions (1) and (2), the following expression (5) is obtained. Additionally, when the expression is rearranged by eliminating k by using the expression (3), the following expression (6) is derived.

S=Ga−kRa (4)

S=Gs+Gn−k(Rs+Rn) (5)

S=Gs−(Gn/Rn)Rs (6)

In this case, the G signal and the R signal have different light absorption characteristics, and Gs>(Gn/Rn)Rs is satisfied. Accordingly, the pulse wave component S from which noise is removed can be calculated by the expression (6).

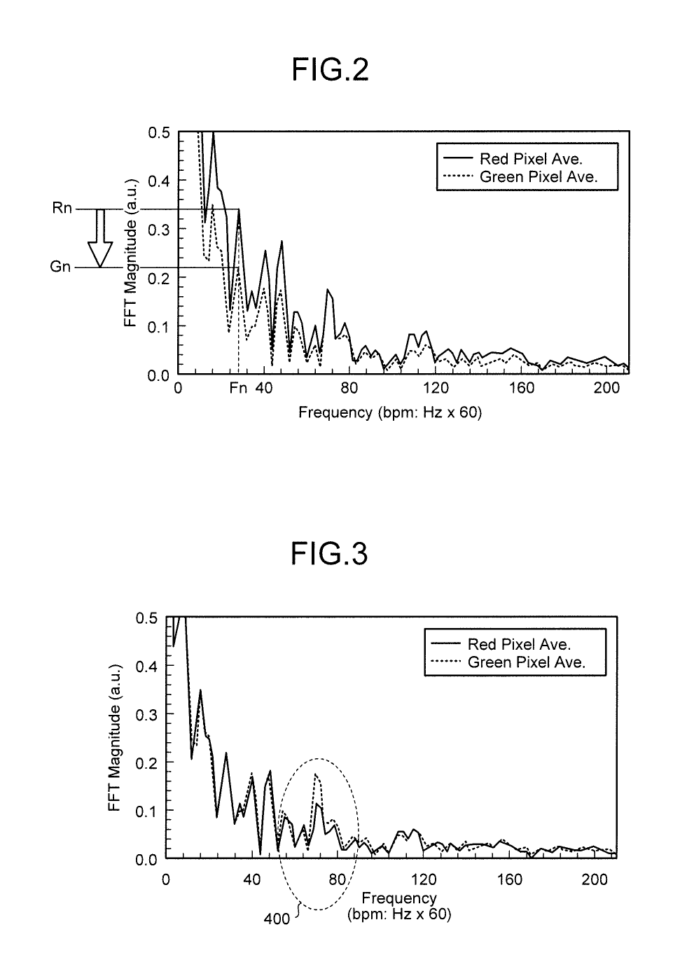

FIG. 2 is a diagram illustrating an example of a spectrum of each of the G signal and the R signal. In the graph illustrated in FIG. 2, the vertical axis indicates signal intensity, and the horizontal axis indicates a frequency (bpm). As illustrated in FIG. 2, sensitivity of the imaging element is different between the G component and the R component, so that the signal intensity is different therebetween. Both in the R component and the G component, noise appears out of the range from 30 bpm to 240 bpm, specifically, in a specific frequency band equal to or larger than 3 bpm and smaller than 20 bpm. Accordingly, as illustrated in FIG. 2, the signal intensity corresponding to a designated frequency Fn included in the specific frequency band equal to or larger than 3 bpm and smaller than 20 bpm can be extracted as Gn and Rn. With Gn and Rn, the correction coefficient k for the difference in sensitivity can be derived.

FIG. 3 is a diagram illustrating an example of the spectrum of each signal of the G component and the R component multiplied by the correction coefficient k. FIG. 3 illustrates an example of a result obtained by multiplying an absolute value of the correction coefficient. Also in the graph illustrated in FIG. 3, the vertical axis indicates the signal intensity, and the horizontal axis indicates the frequency (bpm). As illustrated in FIG. 3, when the correction coefficient k is multiplied by the spectrum of the R signal, the sensitivity is aligned between the components including the G component and the R component. Specifically, the signal intensity in the spectrum in the specific frequency band is substantially the same for the most part. In a peripheral region 400 of a frequency actually including the pulse wave, the signal intensity in the spectrum is not aligned between the G component and the R component.

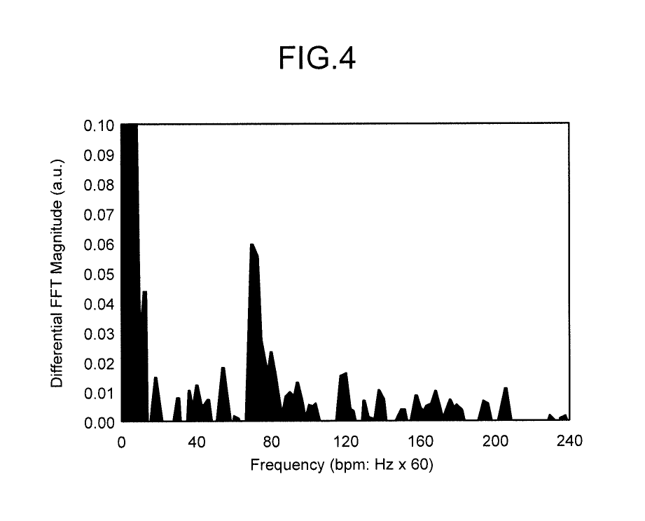

FIG. 4 is a diagram illustrating an example of the spectrum after an arithmetic operation. In FIG. 4, for convenience of explanation, a scale of the signal intensity indicated by the vertical axis is enlarged to improve visibility of the frequency band in which the pulse wave appears. As illustrated in FIG. 4, when the spectrum of the R signal after being multiplied by the correction coefficient k is subtracted from the spectrum of the G signal, it can be seen that the noise component is reduced in a state in which intensity of a signal component in which the pulse wave appears due to a difference in the light absorption characteristic between the G component and the R component is maintained as much as possible. In this way, the waveform of the pulse wave signal from which only the noise component is removed can be detected.

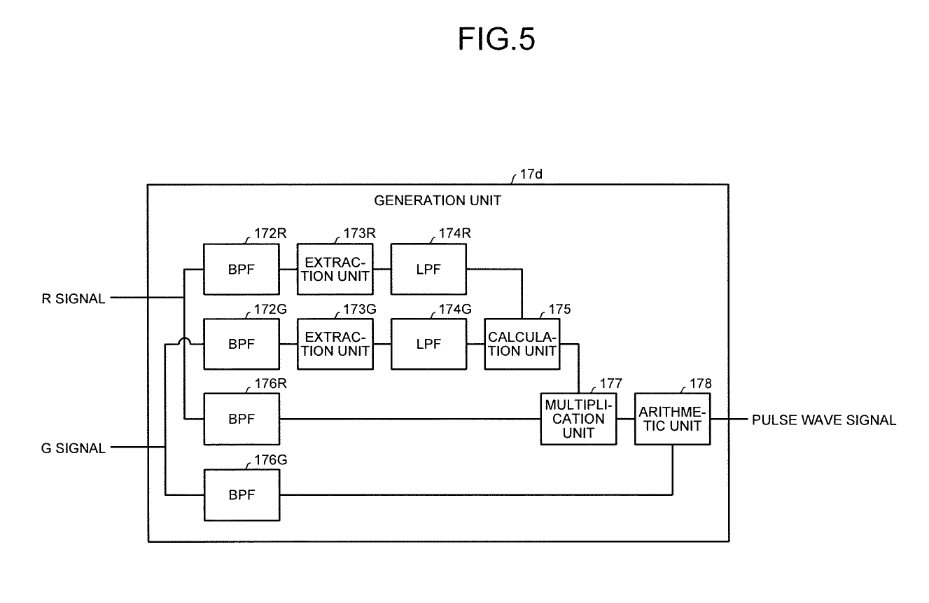

Subsequently, the following specifically describes a functional configuration of the generation unit 17d. FIG. 5 is a block diagram illustrating the functional configuration of the generation unit 17d illustrated in FIG. 1. As illustrated in FIG. 5, the generation unit 17d includes band-pass filters (BPFs) 172R and 172G, extraction units 173R and 173G, low-pass filters (LPFs) 174R and 174G, a calculation unit 175, BPFs 176R and 176G, a multiplication unit 177, and an arithmetic unit 178. FIG. 2 to FIG. 4 illustrate an example of detecting the pulse wave in the frequency region. FIG. 5 illustrates a functional configuration in a case of detecting the pulse wave by canceling the noise component in a time region to reduce a time for conversion into the frequency component.

For example, from the statistical unit 17c to the generation unit 17d, the time series data of the representative value of the R component in the partial image corresponding to the living body region is input as the R signal, and the time series data of the representative value of the G component in the partial image corresponding to the living body region is input as the G signal. Of these, the R signal is input to the BPF 172R and the BPF 176R in the generation unit 17d, and the G signal is input to the BPF 172G and the BPF 176G in the generation unit 17d.

Each of the BPF 172R, the BPF 172G, the BPF 176R, and the BPF 176G is a band-pass filter that passes only a signal component in a predetermined frequency band therethrough and removes a signal component in a frequency band other than the predetermined frequency band. The BPF 172R, the BPF 1726, the BPF 176R, and the BPF 176G may be implemented as hardware, or implemented as software.

The following describes a difference in the frequency band of the signal component that is passed by the BPF. The BPF 172R and the BPF 172G passes the signal component in the specific frequency band in which the noise component more remarkably appears than that in another frequency band.

Such a specific frequency band can be defined by being compared with a frequency band that may be employed by the pulse wave. As an example of the frequency band that may be employed by the pulse wave, exemplified is a frequency band equal to or larger than 0.5 Hz and equal to or smaller than 4 Hz, that is, a frequency band equal to or larger than 30 bpm and equal to or smaller than 240 bpm for one minute. Accordingly, as an example of the specific frequency band, a frequency band smaller than 0.5 Hz and larger than 4 Hz that is difficult to be measured as the pulse wave can be employed. Part of the specific frequency band may be overlapped with the frequency band that may be employed by the pulse wave. For example, a section from 0.7 Hz to 1 Hz that is hardly measured as the pulse wave may be permitted to be overlapped with the frequency band that may be employed by the pulse wave, and the frequency band smaller than 1 Hz and equal to or larger than 4 Hz may be employed as the specific frequency band. The specific frequency band can be narrowed to a frequency band in which noise appears more remarkably by causing the frequency band smaller than 1 Hz and equal to or larger than 4 Hz to be an outer edge. For example, noise appears more remarkably in a low frequency band lower than the frequency band that may be employed by the pulse wave than in a high frequency band higher than the frequency band that may be employed by the pulse wave. Thus, the specific frequency band can be narrowed to a frequency band smaller than 1 Hz. Many differences in sensitivity of the imaging elements of the components are included in the vicinity of a DC component the space frequency of which is zero, so that the specific frequency band can also be narrowed to a frequency band equal to or larger than 0.05 Hz and smaller than 1 Hz. The specific frequency band can also be narrowed to a frequency band equal to or larger than 0.05 Hz and equal to or smaller than 0.3 Hz in which noise easily appears, the noise including flicker of environmental light and the like in addition to motion of a human body such as blinking or swinging of the body.

As an example, the following description will be provided assuming that the BPF 172R and the BPF 172G pass the signal component therethrough, the signal component in the frequency band equal to or larger than 0.05 Hz and equal to or smaller than 0.3 Hz as the specific frequency band. The case of using the band-pass filter to extract the signal component in the specific frequency band is exemplified herein. To extract the signal component in a frequency band smaller than a certain frequency, a low-pass filter can be used.

The BPF 176R and the BPF 176G pass the signal component therethrough, the signal component in the frequency band that may be employed by the pulse wave, for example, the frequency band equal to or larger than 0.5 Hz and equal to or smaller than 4 Hz. Hereinafter, the frequency band that may be employed by the pulse wave may be referred to as a “pulse wave frequency band”.

The extraction unit 173R extracts an absolute intensity value of the signal component of the R signal in the specific frequency band. For example, the extraction unit 173R performs absolute value arithmetic processing on the signal component of the R component in the specific frequency band to extract the absolute intensity value of the signal component in the specific frequency band. The extraction unit 173G extracts the absolute intensity value of the signal component of the G signal in the specific frequency band. For example, the extraction unit 173G performs absolute value arithmetic processing on the signal component of the G component in the specific frequency band to extract the absolute intensity value of the signal component in the specific frequency band.

The LPF 174R and the LPF 174G are low-pass filters that perform smoothing processing on time series data of the absolute intensity value in the specific frequency band to respond to a temporal change. For example, the LPF 174R and the LPF 174G pass the signal component in the frequency band equal to or smaller than 0.1 Hz therethrough. The LPF 174R and the LPF 174G are the same except that a signal input to the LPF 174R is the R signal and a signal input to the LPF 174G is the G signal. Through the smoothing processing, absolute value intensities R′n and G′n in the specific frequency band can be obtained.

The calculation unit 175 performs division “G′n/R′n”, dividing the absolute value intensity G′n of the G signal in the specific frequency band output by the LPF 174G by the absolute value intensity R′n of the R signal in the specific frequency band output by the LPF 174R. In this way, the correction coefficient k for the difference in sensitivity is calculated.

The multiplication unit 177 multiplies the signal component of the R signal in the pulse wave frequency band output by the BPF 176R by the correction coefficient k calculated by the calculation unit 175.

The arithmetic unit 178 performs an arithmetic operation “Gs−k*Rs” of subtracting the signal component of the R signal in the pulse wave frequency band by which the correction coefficient k is multiplied by the multiplication unit 177 from the signal component of the G signal in the pulse wave frequency band output by the BPF 176G. The signal thus obtained corresponds to the pulse wave signal of a face, and a sampling frequency thereof corresponds to a frame frequency at which the image is taken.

The detection unit 17e is a processing unit that detects the pulse wave from the pulse wave signal generated by the generation unit 17d. According to one aspect, the detection unit 17e can directly output the waveform of the pulse wave signal generated by the generation unit 17d as a pulse waveform. According to another aspect, the detection unit 17e can detect the pulse rate from the pulse wave signal generated by the generation unit 17d. For example, as an example of a method for detecting the pulse rate, the detection unit 17e can detect the pulse rate from the spectrum of the pulse wave signal by converting the pulse wave signal of a predetermined time length into a frequency region. In this case, the pulse wave frequency band of the spectrum of the pulse wave signal, that is, a frequency that reaches a peak in a range being equal to or larger than 0.5 Hz and equal to or smaller than 4 Hz can be detected as the pulse rate. As another example of the method for detecting the pulse rate, the detection unit 17e can calculate the pulse rate by performing peak detection, for example, detection of a zero cross point of a differential waveform on the waveform of the pulse wave signal every time when the generation unit 17d generates the pulse wave signal. In this case, when the peak of the waveform of the pulse wave signal is detected through peak detection, the detection unit 17e stores, in an internal memory (not illustrated), a sampling time in which the peak, that is, a local maximum point is detected. Thereafter, when the peak appears, the detection unit 17e obtains a time difference between the peak and the local maximum point previous to the peak by a predetermined parameter n, and can detect the pulse rate by dividing the time difference by n.

The calculation unit 17f is a processing unit that calculates a variation index for evaluating a degree of disturbance of the pulse wave included in the pulse wave signal generated by the generation unit 17d. According to one aspect, the calculation unit 17f calculates five variation indices of the following (1) to (5). For example, the calculation unit 17f calculates (1) a peak ratio, and (2) an area of spectral distribution as the variation indices in the frequency region of the pulse wave signal. The calculation unit 17f also calculates (3) fluctuations in time intervals, (4) a fluctuation in a difference between adjacent extreme values, and (5) a correlation coefficient as the variation indices in the time region of the pulse wave signal. The following sequentially describes a method for calculating the variation indices of (1) to (5) described above.

(1) Peak Ratio

For example, as an example of the peak ratio, the calculation unit 17f can use a ratio between a first peak and a second peak among peaks included in the spectrum of the pulse wave signal.

Specifically, the calculation unit 17f converts the pulse wave signal into the frequency region. In this case, the calculation unit 17f can use an optional conversion method. For example, the calculation unit 17f can apply discrete Fourier transform (DFT), Fourier transform, fast Fourier transform (FFT), discrete cosine transform (DCT), and the like to the conversion method.

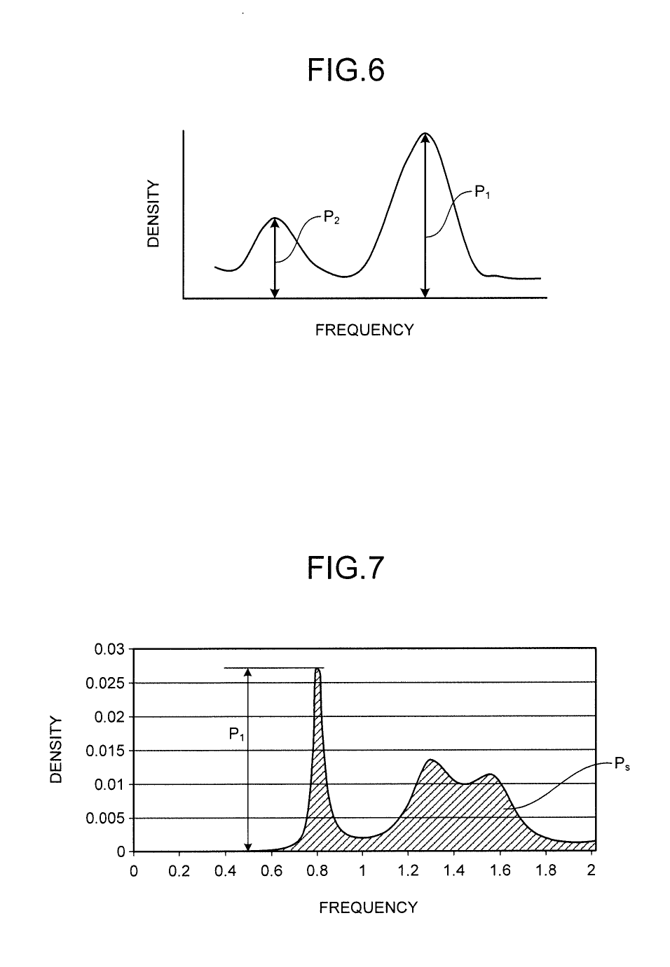

After the pulse wave signal is converted into the frequency region as described above, the calculation unit 17f detects the first peak and the second peak from among the peaks included in the spectrum of the pulse wave signal. FIG. 6 is a diagram illustrating an example of the spectrum of the pulse wave signal. In the graph illustrated in FIG. 6, the vertical axis indicates density, and the horizontal axis indicates a frequency. As illustrated in FIG. 6, when the spectrum is obtained from the pulse wave signal, the calculation unit 17f detects a first peak P₁ having the highest density and a second peak P₂ having the second highest density in the spectrum. Thereafter, as represented by the following expression (7), the calculation unit 17f calculates a peak ratio I₁ by dividing the density at the second peak P₂ by the density at the first peak P₁.

I₁=P₂/P₁ (7)

The peak ratio I₁ is a variation index, and as a value thereof is closer to zero, superimposition of a noise component can be evaluated to be smaller. This is because there is a high probability that the pulse wave is extracted as a main component from the pulse wave signal through the signal generation processing, the first peak corresponds to a component of the pulse wave (signal), and the second peak corresponds to a component of noise.

As the second peak becomes higher, in other words, as the second peak becomes closer to the first peak, a numerator value in the expression (7) increases and a value of the peak ratio I₁ increases. In this way, as the peak ratio I₁ becomes closer to “1”, it can be estimated that a noise component being similar to an actual pulse wave and having a size equivalent to that of the actual pulse wave may be included in the pulse wave signal with high possibility. In this case, a ratio between the noise component and the pulse wave component may be inverted. Also in such a case, by using the peak ratio I₁ for output control, the pulse wave detected from the pulse wave signal is prevented from being output when the noise component having a strength equivalent to that of the pulse wave component is included in the pulse wave signal.

Even when the noise component is spread across a wide band of the pulse wave frequency band, the value of the peak ratio I₁ is decreased if the pulse wave component is sufficiently stronger than the noise component. In this case, by using the peak ratio I₁ for output control, determination can be made to output a detection result of the pulse wave irrespective of an extent of a noise floor.

(2) Area of Spectral Distribution

The calculation unit 17f can use an area of spectral distribution of the pulse wave signal as an example of the area of spectral distribution.

Specifically, similarly to the case of the (1) peak ratio described above, the calculation unit 17f converts the pulse wave signal into the frequency region. FIG. 7 is a diagram illustrating an example of the spectrum of the pulse wave signal. FIG. 7 illustrates a spectrum derived from a pulse wave signal different from the pulse wave signal from which the spectrum illustrated in FIG. 6 is derived. As illustrated in FIG. 7, the calculation unit 17f calculates an area Ps of spectral distribution by integrating the spectrum of the pulse wave signal with a section of the pulse wave frequency band. Thereafter, the calculation unit 17f normalizes the area Ps of spectral distribution obtained through the integration described above with a maximum value P₁ in the section of the pulse wave frequency band. That is, the calculation unit 17f calculates an area I₂ of spectral distribution by the following expression (8).

I₂=(∫P(f)df)/P₁ (8)

It can be seen that the area I₂ of spectral distribution is a variation index, and as a value thereof is closer to zero, superimposition of a noise component can be evaluated to be smaller. This is because only a portion of the pulse wave component appears to be sharply projected in a case of an ideal spectrum of the pulse wave signal, so that it is axiomatic that the area becomes closer to zero when being normalized with the maximum value. The area is increased as the noise component appears across a wide range of the pulse wave frequency band and the density of the noise floor is increased, so that the value of the area I₂ of spectral distribution is also increased. Also in such a case, an output can be suppressed by using the area I₂ of spectral distribution for output control.

(3) Fluctuations in Time Interval

As an example of fluctuations in time intervals, the calculation unit 17f can calculate time intervals between intersection points of the waveform of the pulse wave signal and a plurality of straight lines parallel with a time axis to use a standard deviation of the time intervals.

As described above, to obtain the time intervals between the intersection points, the time intervals between the intersection points can be obtained for all the intersection points at which the waveform intersects with the straight line. However, when the waveform of the pulse wave signal does not approximate to a sin wave and continuously takes extreme values in a time shorter than a period of the pulse wave, for example, noise having a higher frequency than that of the pulse wave may be mixed. In this case, fluctuations in the time intervals between the intersection points are reduced, so that the noise may be accidentally evaluated to be small. To prevent such a situation, the time interval between the intersection points may be obtained for any one of an intersection point of a rising part of the waveform and the straight line, and an intersection point of a falling part of the waveform and the straight line among the intersection points at which the waveform intersects with the straight line. The following exemplifies a case of obtaining the time interval between the intersection points for the intersection point of the falling part of the waveform and the straight line. Alternatively, the time interval between the intersection points may be obtained for all the intersection points, or the time interval between the intersection points may be obtained for the intersection point of the rising part of the waveform and the straight line.

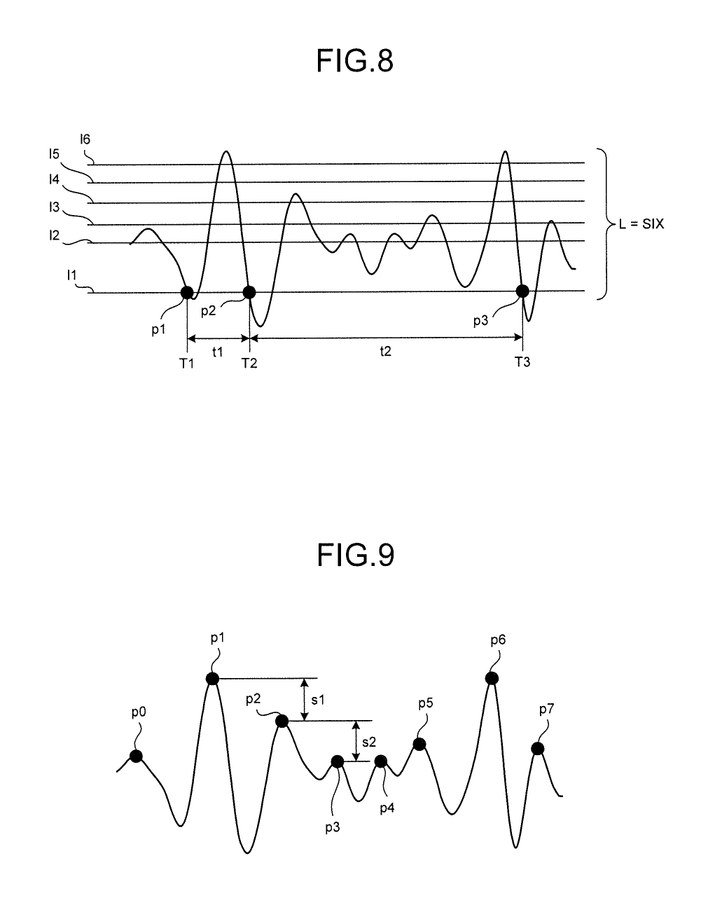

For example, the calculation unit 17f specifies the intersection point of the waveform of the pulse wave signal and each of a plurality of straight lines L₁ to L_{L} parallel with the time axis for each straight line. FIG. 8 is a diagram illustrating an example of the waveform of the pulse wave signal. FIG. 8 illustrates six straight lines l₁ to l₆ parallel with the time axis together with the waveform of the pulse wave signal. As illustrated in FIG. 8, the calculation unit 17f specifies intersection points p1, p2, and p3 at which the straight line l₁ intersects with the falling part of the waveform of the pulse wave signal. The calculation unit 17f calculates a difference between a time T1 at the intersection point p1 and a time T2 at the intersection point p2, that is, T2−T1 to calculate a time interval t1. The calculation unit 17f also calculates a difference between the time T2 at the intersection point p2 and a time T3 at the intersection point p3, that is, T3−T2 to calculate a time interval t2. Thereafter, the calculation unit 17f calculates a standard deviation σ of the time intervals in the straight line l₁ according to the following expression (9) by using the time interval t1 and the time interval t2 of the straight line l₁, and an average value t_{avg} of the time interval. Similarly, according to the following expression (9), the calculation unit 17f calculates the standard deviation of the time intervals for the straight lines l₂ to l₆. Subsequently, the calculation unit 17f calculates a fluctuation I₃ in time intervals by summing up standard deviations of the time intervals of the straight lines l₁ to l₆ according to the following expression (10). In the following expression (9), “tᵢ” indicates the i-th time interval, and “n” indicates the number of intersection points.

$$\begin{matrix}{σ}^{( l )} = \sqrt{\frac{1}{n - 1} {\sum }^{n} ( {t}_{i} - {t}_{avg} )} & ( 9 ) \\ {I}_{3} = {\sum }_{l = 1}^{L} {σ}^{( l )} & ( 10 )\end{matrix}$$

It can be seen that the fluctuation I₃ in time intervals is a variation index, and as a value thereof is closer to zero, superimposition of a noise component can be evaluated to be smaller. This is because the time intervals become substantially regular intervals in a case of an ideal pulse wave signal, so that the value becomes closer to zero. When magnitude of amplitude of the pulse waveform becomes unstable due to the noise component, the value of the fluctuation I₃ in time intervals is increased. Also in such a case, an output can be suppressed by using the fluctuation I₃ in time intervals for output control.

Exemplified is a case of summing up the standard deviations of the time intervals of the straight lines l₁ to l_{L}. Alternatively, various pieces of statistical processing other than summing up can be performed on the standard deviation of the time intervals obtained for each straight line. For example, the fluctuation I₃ in time intervals may be calculated by averaging the standard deviations of the time intervals of the straight lines l₁ to l_{L}, or a median of the standard deviations of the time intervals of the straight lines l₁ to l_{L} may be caused to be the fluctuation I₃ in time intervals.

An upper limit value or a lower limit value of the amplitude taken by the waveform of the pulse wave signal, that is, the waveform close to what is called a sin wave is assumed to be fluctuated depending on a period of the waveform. In this case, in a certain period, the upper limit value of the amplitude may be reduced, or the lower limit value of the amplitude may be increased. In this case, there may be a situation in which the straight line passing through near upper and lower ends of the waveform among the straight lines l₁ to l_{L} intersects with the waveform in a certain period, but is difficult to intersect with the waveform in another period. Due to this, the calculation unit 17f gives larger weight to the straight line l_{c} passing through near the center of the waveform than to the straight line passing through near the upper and lower ends of the waveform among the standard deviations of the time intervals of the straight lines l₁ to l_{L}. Thereafter, the calculation unit 17f can calculate the fluctuation I₃ in time intervals by performing weighted average on the standard deviations of the time intervals of the straight lines l₁ to l_{L}. Accordingly, even when a local fluctuation is caused in the upper limit value and the lower limit value of the amplitude of the waveform of the pulse wave signal, the value of the variation index can be prevented from being excessively increased.

(4) Fluctuation in Difference Between Adjacent Extreme Values

As an example of a fluctuation in a difference between adjacent extreme values, the calculation unit 17f can calculate a difference in amplitude between adjacent extreme values in the waveform of the pulse wave signal, and can use a standard deviation of the difference in amplitude. As described above, when the difference in amplitude is obtained, as an example, any of a difference in amplitude between local maximum values and a difference in amplitude between local minimum values can be obtained. The following exemplifies a case of obtaining the difference in amplitude between the local maximum values. Alternatively, the difference in amplitude between local minimum values may be obtained.

Specifically, the calculation unit 17f detects the local maximum point in the waveform of the pulse wave signal. The local maximum point can be specified by detecting the zero cross point of the differential waveform of the pulse wave signal. Thereafter, the calculation unit 17f calculates a difference in amplitude between local maximum points. FIG. 9 is a diagram illustrating an example of the waveform of the pulse wave signal. FIG. 9 exemplifies the waveform of the pulse wave signal different from that in FIG. 8, and illustrates an example of a case in which eight local maximum points p0 to p7 are detected. As illustrated in FIG. 9, a difference in amplitude between the local maximum point p1 and the local maximum point p2 in the waveform of the pulse wave signal is calculated to be “s1”, and a difference in amplitude between the local maximum point p2 and the local maximum point p3 is calculated to be “s2”. Thereafter, by using differences in amplitude between the local maximum points and an average value of the differences in amplitude, the calculation unit 17f calculates standard deviations of the differences in amplitude between the local maximum points p0 to p7 according to the following expression (11). The calculation unit 17f then sums up the standard deviations of the differences in amplitude between the extreme values according to the following expression (12) to calculate a fluctuation I₄ in a difference between adjacent extreme values.

$$\begin{matrix}{σ}^{( m )} = \sqrt{\frac{1}{n - 1} {\sum }^{n} ( {s}_{i} - {s}_{avg} )} & ( 11 ) \\ {I}_{4} = {\sum }_{m = 1}^{M} {σ}^{( m )} & ( 12 )\end{matrix}$$

In the above expression (11), “sᵢ” indicates the i-th difference in amplitude, and “n” indicates the number of local maximum points or local minimum points. In the above expression (12), “m” indicates the number of types of extreme values, for example, two types including the local maximum value and the local minimum value. That is, to obtain only a difference in amplitude between the local maximum values as extreme value points, the standard deviation of the differences in amplitude between the local maximum points calculated by the above expression (11) can be directly caused to be the fluctuation I₄ in a difference between adjacent extreme values. To also obtain the difference in amplitude between the local minimum values as the extreme value points, the sum of standard deviations of both may be calculated as the fluctuation I₄ in a difference between adjacent extreme values.

It can be seen that the fluctuation I₄ in a difference between adjacent extreme values is also a variation index, and as the value thereof is closer to zero, superimposition of a noise component can be evaluated to be smaller. This is because the local maximum value and the local minimum value of amplitude are substantially the same in respective periods in a case of an ideal pulse wave signal, so that the difference between the adjacent extreme values becomes closer to zero. When the local maximum value or the local minimum value of the amplitude of the pulse waveform becomes unstable due to the noise component, the value of the fluctuation I₄ in a difference between adjacent extreme values is increased. Also in such a case, an output can be suppressed by using the fluctuation I₄ in a difference between adjacent extreme values for output control.

(5) Correlation Coefficient

As an example of a correlation coefficient, the calculation unit 17f may employ an autocorrelation method of shifting, between the waveform of the pulse wave signal and a duplicated waveform obtained by duplicating part of the former waveform in a predetermined window width, the duplicated waveform to calculate correlation coefficients therebetween, and can use the maximum value of the correlation coefficients.

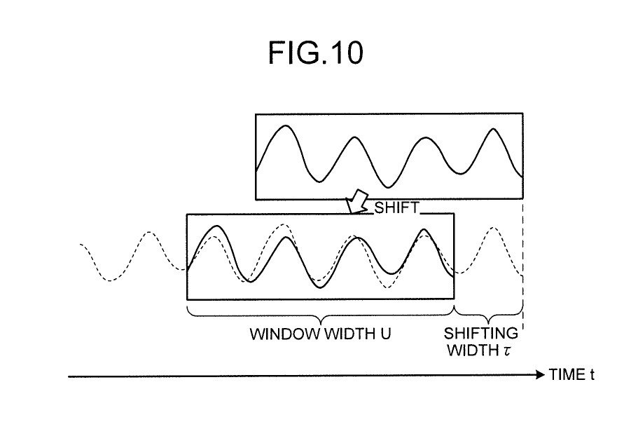

FIG. 10 is a diagram illustrating an example of the waveform of the pulse wave signal. As illustrated in FIG. 10, the calculation unit 17f duplicates the waveform corresponding to a portion defined with a predetermined window width U in the waveform of the pulse wave signal. The calculation unit 17f then causes the thus obtained duplicated waveform of the window width U to shift frontward on the time axis across a shifting width τ, and calculates a correlation coefficient cor between the waveform of the pulse wave signal and the duplicated waveform according to the following expression (13). In the following expression (13), “x” indicates time series data of amplitude of the duplicated waveform, and “y” indicates time series data of amplitude of the waveform of the pulse wave signal as a detection target. In the following expression (13), a bar added to each of “x” and “y” indicates an average value thereof. Thereafter, the calculation unit 17f causes the duplicated waveform to shift frontward on the time axis by updating the shifting width τ, and repeatedly calculates the correlation coefficient. The maximum value of the thus obtained correlation coefficient can be used as the correlation coefficient I₅.

$$cor = \frac{{\sum }_{i = 1}^{n} ( {x}_{i} - \overline{x} ) ( {y}_{i} - \overline{y} )}{\sqrt{{\sum }_{i = 1}^{n} {( {x}_{i} - \overline{x} )}^{2}} \sqrt{{\sum }_{i = 1}^{n} {( {y}_{i} - \overline{y} )}^{2}}} \qquad ( 13 )$$

It can be seen that the correlation coefficient I₅ is a variation index, and as the value thereof is closer to one, superimposition of a noise component can be evaluated to be smaller. This is because the pulse wave has periodicity in a case of an ideal pulse wave signal, so that the maximum value of the correlation coefficient calculated by the autocorrelation method becomes closer to “1”. When the waveform of the pulse wave signal is disturbed due to the noise component, the periodicity thereof is lowered, so that the value of the correlation coefficient I₅ is reduced. Also in such a case, an output can be suppressed by using the correlation coefficient I₅ for output control.

The indices related to the time region according to (3) to (5) have an advantage that quality of the pulse wave signal can be determined with higher accuracy even when the time length of the pulse wave signal is short as compared with the index related to the frequency region.

Returning to FIG. 1, the output control unit 17g is a processing unit that perform output control of the pulse wave signal generated by the generation unit 17d by using the variation index calculated by the calculation unit 17f.

According to one aspect, the output control unit 17g can obtain a total variation index I_{T} totalizing the five variation indices I₁ to I₅ by giving predetermined weights m₁ to m₅ to the variation indices I₁ to I₅ calculated by the calculation unit 17f and performing weighted average on the variation indices I₁ to I₅ in accordance with each weight. In this way, to obtain the total variation index I_{T}, weighted average is performed after the respective variation indices I₁ to I₅ are normalized. For example, normalization is implemented by matching scales of values of the variation indices I₁ to I₅ with each other or taking an inverse number of the variation index I₅. By way of example, the weights m₁ to m₅ can be calculated in advance by using various learning methods such as boosting, a neural network, and a support vector machine, or can be optionally set by a developer and the like of the signal processing program described above.

Thereafter, the output control unit 17g determines whether the total variation index I_{T} is smaller than a predetermined threshold TH. If the total variation index I_{T} is not smaller than the threshold TH, it can be estimated that the noise component superimposed on the pulse wave signal generated by the generation unit 17d is large, which hinders a detection result of the pulse wave. In this case, the output control unit 17g suppresses an output of the detection result of the pulse wave detected by the detection unit 17e. If the total variation index I_{T} is smaller than the threshold TH, it can be estimated that the noise component superimposed on the pulse wave signal generated by the generation unit 17d is small, which hardly hinders the detection result of the pulse wave. In this case, the output control unit 17g causes the detection result of the pulse wave detected by the detection unit 17e to be output to a predetermined output destination.

When the detection result of the pulse wave, for example, the pulse rate or the pulse waveform is output as described above, they can be output to an optional output destination including the touch panel 13 included in the pulse wave detection device 10. For example, when a diagnostic program for diagnosing an operation of an autonomic nerve based on the pulse rate or fluctuations in a pulse period or diagnosing a heart disorder and the like based on the pulse waveform is installed in the pulse wave detection device 10, the diagnostic program can be caused to be the output destination. A server device and the like providing the diagnostic program as a Web service may be caused to be the output destination. Additionally, a terminal device used by a person relevant to a user utilizing the pulse wave detection device 10, for example, a caregiver or a doctor can be caused to be the output destination. Due to this, a monitoring service can be provided outside a hospital, for example, at home or at one's desk. Obviously, a measurement result or a diagnostic result of the diagnostic program can also be displayed on the terminal device of a relevant person including the pulse wave detection device 10.

The signal processing unit 17 can be implemented by causing a central processing unit (CPU) or a micro processing unit (MPU) to execute the signal processing program. The functional units described above can be implemented with hard wired logic such as an application specific integrated circuit (ASIC) and a field programmable gate array (FPGA).

As a memory used by the signal processing unit 17, a semiconductor memory element or a storage device can be employed. Examples of the semiconductor memory element include a flash memory, a dynamic random access memory (DRAM), and a static random access memory (SRAM). Examples of the storage device include a storage device such as a hard disk and an optical disc.

Processing Procedure

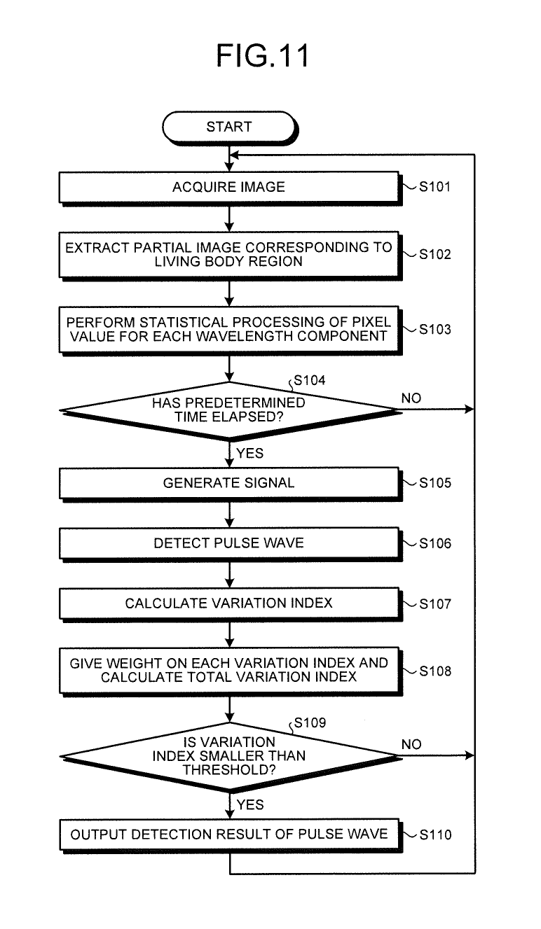

FIG. 11 is a flowchart illustrating a signal processing procedure according to the first embodiment. The signal processing is repeatedly performed when the signal processing program is started through an operation on the touch panel 13 and the like or operates in a background. When an interrupting operation is received via the touch panel 13 and the like, the signal processing can be stopped.

As illustrated in FIG. 11, when the acquisition unit 17a acquires an image (Step S101), the extraction unit 17b extracts a predetermined face part, for example, a partial image corresponding to a face region based on the nose of the subject from the image acquired at Step S101 (Step S102). Thereafter, the statistical unit 17c outputs, to the generation unit 17d, time series data of the representative value of each pixel included in the partial image of the face region extracted at Step S102 for each of the R component and the G component (Step S103).

Thereafter, if the time series data of the R component and the G component is accumulated for a predetermined time (Yes at Step S104), the generation unit 17d performs processing as described below. That is, the generation unit 17d generates a signal in which components in a specific frequency band other than the pulse wave frequency band are canceled with each other between the R component and the G component (Step S105). Subsequently, the detection unit 17e detects the pulse wave, for example, the pulse rate or the pulse waveform from the pulse wave signal generated at Step S105 (Step S106).

The calculation unit 17f then calculates the variation indices I₁ to I₅ by using the pulse wave signal generated at Step S105 (Step S107). Thereafter, the output control unit 17g gives predetermined weights m₁ to m₅ to the variation indices I₁ to I₅ calculated by the calculation unit 17f, and performs weighted average on the variation indices I₁ to I₅ according to the respective weights to obtain the total variation index I_{T} (Step S108).

The output control unit 17g determines whether the total variation index I_{T} calculated at Step S108 is smaller than the predetermined threshold TH (Step S109). If the total variation index I_{T} is not smaller than the threshold TH (No at Step S109), it can be estimated that the noise component superimposed on the pulse wave signal generated at Step S105 is large, which hinders the detection result of the pulse wave. In this case, the process returns to Step S101 without outputting the detection result of the pulse wave detected by the detection unit 17e.

If the total variation index I_{T} is smaller than the threshold TH (Yes at Step S109), it can be estimated that the noise component superimposed on the pulse wave signal generated at Step S105 is small, which hardly hinders the detection result of the pulse wave. In this case, the output control unit 17g causes the detection result of the pulse wave detected by the detection unit 17e to be output to a predetermined output destination (Step S110), and the process proceeds to Step S101.

### Advantageous Effects of First Embodiment

As described above, the pulse wave detection device 10 according to the present embodiment calculates the variation index for evaluating a degree of disturbance of the pulse wave based on the pulse wave signal generated from the living body image, and controls whether to output the pulse wave signal by using the variation index. Thus, when a noise removal function does not work because the noise at a level at which the component corresponding to the pulse wave is difficult to be extracted is superimposed on the signal, the pulse wave detection device 10 according to the present embodiment can suppress the output of the detection result of the pulse wave. The pulse wave detection device 10 according to the present embodiment can also suppress the output of the detection result of the pulse wave when the noise component having a period similar to that of the pulse wave is superimposed on the pulse wave signal due to evaluation of the pulse wave signal with the variation index. Accordingly, the pulse wave detection device 10 according to the present embodiment can appropriately perform output control of the detection result of the pulse wave.

The pulse wave detection device 10 according to the present embodiment controls whether to output the pulse wave signal by using a plurality of variation indices. Thus, the pulse wave detection device 10 according to the present embodiment can versatilely evaluate the quality of the pulse wave signal. That is, the pulse wave detection device 10 according to the present embodiment can evaluate the quality of the pulse wave signal while compensating weak points with each other between the variation indices. Accordingly, the pulse wave detection device 10 according to the present embodiment can further optimize output control of the detection result of the pulse wave.

### [b] Second Embodiment

The first embodiment has exemplified a case of obtaining the total variation index I_{T} from the five variation indices I₁ to I₅. However, the total variation index I_{T} is not necessarily obtained. By way of example, a second embodiment exemplifies a case of classifying the pulse wave signal into two classes of good and poor by using a classification tree using the variation index as a node.

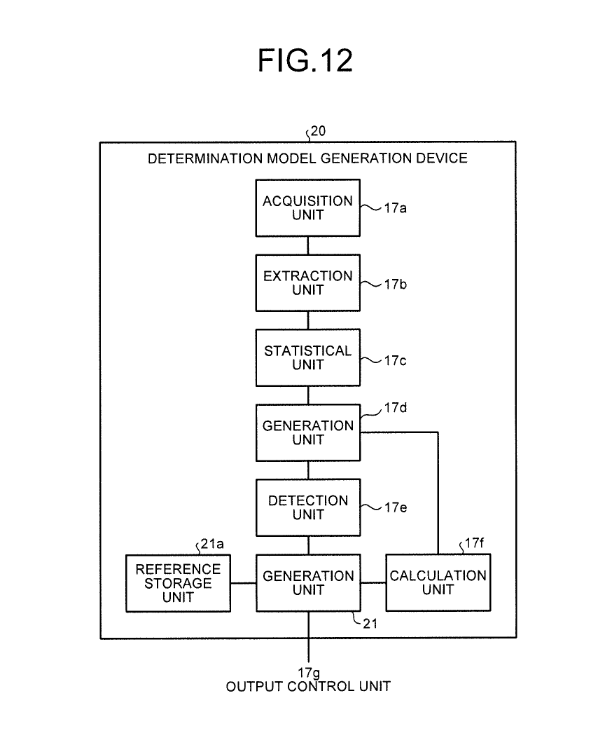

FIG. 12 is a block diagram illustrating a functional configuration of a determination model generation device according to the second embodiment. A determination model generation device 20 illustrated in FIG. 12 generates a determination model of a classification tree using the variation index as a node, a threshold used for determining the quality of the pulse wave signal at the node, and the like. By way of example, the determination model generation device 20 generates the determination model of the classification tree, the threshold, and the like before shipping the pulse wave detection device 10, and sets the determination model to the pulse wave detection device 10. Thus, similarly to the pulse wave detection device 10, functional units of the determination model generation device 20 may be mounted on a portable terminal device, or mounted on a stand-alone computer and the like that sets a parameter to a portable terminal device to be shipped.

As illustrated in FIG. 12, the determination model generation device 20 includes the acquisition unit 17a, the extraction unit 17b, the statistical unit 17c, the generation unit 17d, the detection unit 17e, the calculation unit 17f, a reference storage unit 21a, and a generation unit 21. In FIG. 12, a functional unit that exhibits the same function as that of the functional unit illustrated in FIG. 1 is denoted by the same reference numeral, and description thereof will not be repeated.

Of these, the reference storage unit 21a is a storage unit that stores a reference of the pulse wave signal generated by the generation unit 17d. An example of such a reference includes an electrocardiographic signal obtained by an electrocardiographic sensor that operates in synchronization with the image acquired by the acquisition unit 17a.

The generation unit 21 is a processing unit that generates the determination model of the classification tree using the variation index as a node, the threshold used for determining the quality of the pulse wave signal at the node, and the like. According to one aspect, by using an error between the pulse wave signal generated by the generation unit 17d and the electrocardiographic signal stored in the reference storage unit 21a as a reference, the generation unit 21 generates the determination model including the classification tree and the threshold with which the highest percentage of correct answers can be obtained when the pulse wave signal is classified into two classes of good and poor based on the variation index calculated by the calculation unit 17f. Thereafter, the generation unit 21 sets the previously generated determination model to the output control unit 17g.

Specifically, the generation unit 21 refers to the electrocardiographic signal stored in the reference storage unit 21a as a reference, classifies a pulse wave signal the error of which is within a predetermined range, for example, N beats/minute as “good” from among the pulse wave signals generated by the generation unit 17d, and classifies a pulse wave signal the error of which is out of the predetermined range as “poor”. As an example of a standard of the error for classification, 5 beats/minute can be used. Thereafter, the generation unit 21 learns a determination model applied to classification processing for classifying the pulse wave signal generated by the generation unit 17d into any of two classes of “good” and “poor” by using each variation index calculated by the calculation unit 17f. For machine learning for such classification, an optional algorithm such as boosting, a neural network, and a support vector machine may be employed. By way of example, the following describes a case of generating the classification tree using each variation index as a node. In this case, the generation unit 21 generates, for example, the classification tree by determining a variation index to be employed as a node from among the variation indices I₁ to I₅, a hierarchy in which the node is arranged, and the threshold set to each node so that the percentage of correct answers of classification is the highest. The generation unit 21 then sets, to the output control unit 17g, the generated classification tree and the threshold used for determination at the node of the classification tree, that is, a learning result of the determination model.

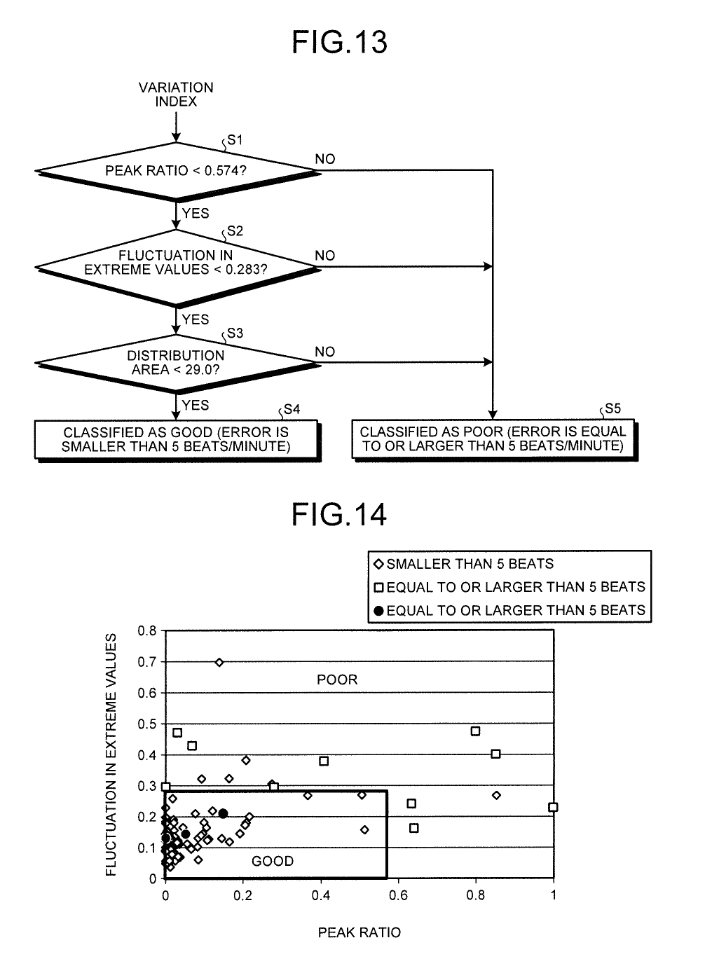

FIG. 13 is a diagram illustrating an example of the determination model. FIG. 13 illustrates an example of the determination model in a case in which the pulse wave signal the error of which is smaller than “5 beats/minute” is classified into the class of “good”, and the pulse wave signal the error of which is equal to or larger than “5 beats/minute” is classified into the class of “poor”. When the determination model illustrated in FIG. 13 is used by the output control unit 17g of the pulse wave detection device 10, the following determination is performed.

As illustrated in FIG. 13, when the calculation unit 17f of the pulse wave detection device 10 calculates each variation index, the output control unit 17g determines whether the peak ratio I₁ is smaller than the threshold “0.574” (Step S1). Subsequently, if the peak ratio I₁ is smaller than the threshold “0.574” (Yes at Step S1), the output control unit 17g further determines whether the fluctuation I₄ in a difference between adjacent extreme values is smaller than the threshold “0.283” (Step S2). If the fluctuation I₄ in a difference between adjacent extreme values is smaller than the threshold “0.283”, the output control unit 17g further determines whether the area I₂ of spectral distribution is smaller than the threshold “29.0” (Step S3). In this case, when the area I₂ of spectral distribution is also smaller than the threshold “29.0”, the pulse wave signal generated by the generation unit 17d is classified into the class of “good” (Step S4). If the peak ratio I₁ is not smaller than the threshold “0.574”, if the fluctuation I₄ in a difference between adjacent extreme values is not smaller than the threshold “0.283”, or if the area I₂ of spectral distribution is not smaller than the threshold “29.0” (No at Step S1, No at Step S2, or No at Step S3), the pulse wave signal generated by the generation unit 17d is classified into the class of “poor” (Step S5).

In this way, the determination model for performing quantitative evaluation can be generated by converting the problem that what weight is given to the variation index for performing classification into two classes into a problem of performing clustering with the error for classification of the quality of the pulse wave signal.

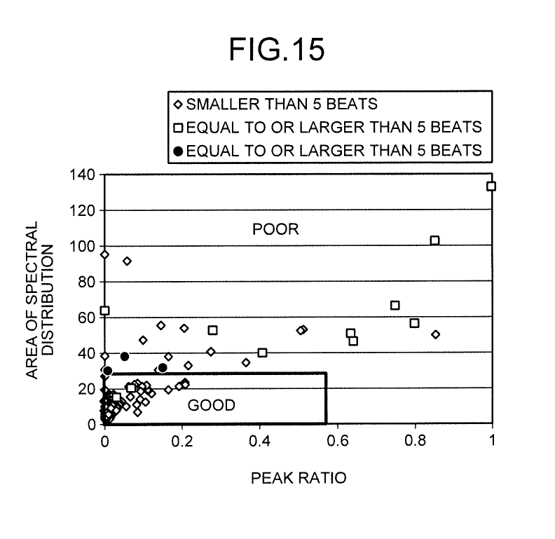

With reference to FIGS. 14 and 15, the following describes determination accuracy of classification. FIG. 14 is a diagram illustrating an example of a classification result based on the fluctuation in a difference between adjacent extreme values and the peak ratio, and FIG. 15 is a diagram illustrating an example of a classification result based on the area of spectral distribution and the peak ratio. FIGS. 14 and 15 illustrate a case in which the pulse wave signal is classified into the classes of “good” and “poor” according to the determination model illustrated in FIG. 13. As an example, a measurement condition of the graph illustrated in FIGS. 14 and 15 is such that the number of persons to be evaluated is 5, duration of the waveform is 15 seconds, a case in which the device is vibrated and a case in which the device is not vibrated are both included, and the number of times of measurement is 90 in total.

For convenience of explanation, FIGS. 14 and 15 illustrate a case in which projection is performed from a three-dimensional space including the peak ratio, the fluctuation in a difference between adjacent extreme values, and the area of spectral distribution to a plane including the fluctuation in a difference between adjacent extreme values and the peak ratio, and a plane including the area of spectral distribution and the peak ratio. In FIGS. 14 and 15, a plot of “⋄” indicates the pulse wave signal the error of which is smaller than 5 beats/minute, and plots of “□” and “•” each indicate the pulse wave signal the error of which is equal to or larger than 5 beats/minute. A thick-line frame illustrated in FIGS. 14 and 15 indicates a boundary between “good” and “poor” specified with the threshold used for the node in the classification tree of the determination model illustrated in FIG. 13.

When the pulse wave signal is classified into the two classes of “good” and “poor” according to the determination model illustrated in FIG. 13, it can be seen that a favorable result can be obtained as illustrated in FIGS. 14 and 15. For example, as illustrated in FIG. 14, there is only a case in which three plots of “•” are classified as “good”, as plots the error of which is equal to or larger than “5 beats/minute”, through threshold determination based on the fluctuation in a difference between adjacent extreme values and the peak ratio. The other plots of “□” are all classified as “poor” through threshold determination based on the fluctuation in a difference between adjacent extreme values and the peak ratio. As illustrated in FIG. 15, it can be seen that the three plots of “•” classified as “good” only through determination based on the fluctuation in a difference between adjacent extreme values and the peak ratio can be classified as “poor” through threshold determination based on the area of spectral distribution and the peak ratio. It can also be seen that the two plots of “□” classified as “good” through threshold determination based on the area of spectral distribution and the peak ratio can be classified as “poor” through threshold determination based on the fluctuation in a difference between adjacent extreme values and the peak ratio.

Processing Procedure

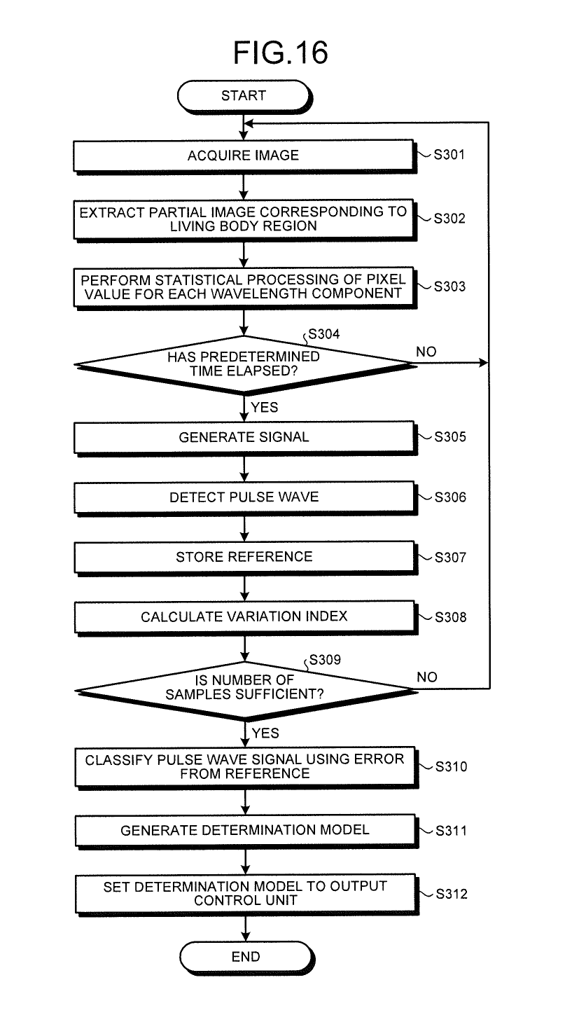

FIG. 16 is a flowchart illustrating a setting processing procedure of the determination model according to the second embodiment. The processing is started when the acquisition unit 17a acquires the image.

As illustrated in FIG. 16, when the acquisition unit 17a acquires the image (Step S301), the extraction unit 17b extracts a predetermined face part, for example, a partial image corresponding to a face region based on the nose of the subject from the image acquired at Step S301 (Step S302). Thereafter, the statistical unit 17c outputs, to the generation unit 17d, time series data of the representative value of each pixel included in the partial image of the face region extracted at Step S302 for each of the R component and the G component (Step S303).

Thereafter, if the time series data of the R component and the G component is accumulated over a predetermined time (Yes at Step S304), the generation unit 17d performs processing as described below. That is, the generation unit 17d generates a signal in which components in the specific frequency band other than the pulse wave frequency band are canceled with each other between the R component and the G component (Step S305). Subsequently, the detection unit 17e detects the pulse wave, for example, the pulse rate or the pulse waveform from the pulse wave signal generated at Step S305 (Step S306).

An electrocardiographic waveform that is obtained in synchronization with the pulse wave signal from which the pulse wave is detected at Step S306 as described above is stored in the reference storage unit 21a as a reference (Step S307). Thereafter, the calculation unit 17f calculates the variation indices I₁ to I₅ by using the pulse wave signal generated at Step S305 (Step S308).

If the number of samples of the pulse wave signal becomes sufficient (Yes at Step S309), the generation unit 21 refers to the electrocardiographic signal stored in the reference storage unit 21a at Step S307, and classifies the pulse wave signal generated at Step S305 into the classes of “good” and “poor” (Step S310).

Thereafter, the generation unit 21 generates the determination model including the classification tree and the threshold with which the highest percentage of correct answers can be obtained when the pulse wave signal is classified into the two classes of good and poor based on the variation index calculated at Step S308 by using the error between the pulse wave signal generated at Step S305 and the electrocardiographic signal stored in the reference storage unit 21a at Step S307 (Step S311). Subsequently, the generation unit 21 sets the determination model generated at Step S311 to the output control unit 17g (Step S312), and ends the process.

### Advantageous Effects of Second Embodiment

As described above, by using the error of the pulse wave signal with respect to the reference, the determination model generation device 20 according to the present embodiment generates the determination model including the classification tree using the variation index as a node and the threshold used for determining the quality of the pulse wave signal at the node. Accordingly, the determination model generation device 20 according to the present embodiment can generate the determination model that can quantitatively evaluate the quality of the pulse wave signal. When output control of the pulse wave signal is performed by using the determination model described above, favorable accuracy in output control can be expected as described with reference to FIGS. 14 and 15.

### [c] Third Embodiment

The embodiments of the disclosed device have been described above. Alternatively, the present invention can be implemented in various different forms other than the embodiments described above. The following describes other embodiments encompassed by the present invention.

### First Modification

The first embodiment exemplifies a case of generating the pulse wave signal in which components in the specific frequency band other than the pulse wave frequency band are canceled with each other between the R component and the G component. Alternatively, the pulse wave signal can be generated by another method. For example, the generation unit 17d may cause time series data obtained by averaging luminance values of G components of the pixels included in the partial image corresponding to the living body region, that is, the G signal to be the pulse wave signal. Although the case of using the G signal as the pulse wave signal is exemplified herein, the R signal or a B signal may be used as the pulse wave signal.

### Second Modification

The first embodiment exemplifies a case in which the total variation index I_{T} is obtained from the variation indices I₁ to I₅. However, the total variation index I_{T} is not necessarily obtained. For example, a threshold is set to each of the variation indices I₁ to I₅. Thereafter, the output control unit 17g can cause the pulse wave signal to be output only when the variation indices I₁ to I₄ are all smaller than the threshold and the variation index I₅ is equal to or larger than the threshold, that is, when all the variation indices satisfy the condition. Alternatively, the output control unit 17g can cause the pulse wave signal to be output when the number of variation indices satisfying the condition is larger than the other variation indices by a majority vote.

### Third Modification

The first embodiment and the second embodiment exemplify a case of using two types of input signals, that is, the R signal and the G signal to detect the pulse wave. Alternatively, an optional number of types of signals and an optional number of signals can be used as input signals so long as the signals have a plurality of different light wavelength components. For example, among signals having different light wavelength components such as R, G, B, IR, and NIR, an optional combination of two signals may be used, or three or more signals may be used.

### First Application Example

For example, the pulse wave detection device 10 may further calculate variation indices from sensor values obtained by various sensors, and may use the variation indices together with the variation indices I₁ to I₅ to determine the quality of the pulse wave signal. Examples of such sensors include a touch sensor, an illuminance sensor, and a distance sensor in addition to a motion sensor such as an acceleration sensor, a gyro sensor, and a pedometer. For example, the variation index can be calculated by using the motion sensor as follows. That is, the number of times when the sensor value obtained by the motion sensor exceeds a predetermined threshold in a predetermined time length can be calculated as the variation index. In a case of the touch sensor, the number of times of touch operation on the touch panel 13 can be calculated as the variation index. In a case of the illuminance sensor, a change amount of illuminance in a predetermined time length can be calculated as the variation index. In a case of the distance sensor, the number of times when a distance between the touch panel 13 and the user's face deviates from a predetermined proper distance can be calculated as the variation index.

Distribution and Integration

The components of the devices illustrated in the drawings are not necessarily physically configured as illustrated. That is, specific forms of distribution and integration of the devices are not limited to those illustrated in the drawings. All or part thereof may be functionally or physically distributed/integrated in arbitrary units depending on various loads or usage states. For example, the first embodiment exemplifies a case in which the pulse wave detection device 10 performs the signal processing described above on a stand-alone basis, but the pulse wave detection device 10 may be implemented as a client server system. For example, the pulse wave detection device 10 may be implemented as a Web server that executes signal processing, or may be implemented as a cloud that provides a service including a signal processing service by outsourcing. In this way, when the pulse wave detection device 10 operates as a server device, a portable terminal device such as a smartphone or a mobile phone and an information processing device such as a personal computer can be accommodated as a client terminal. A pulse wave detection service and a diagnosis service can be provided by performing signal processing when an image reflecting the face of the subject is acquired from the client terminal via a network, and giving a detection result thereof or a diagnostic result obtained by using the detection result to the client terminal as a response.

Signal Processing Program

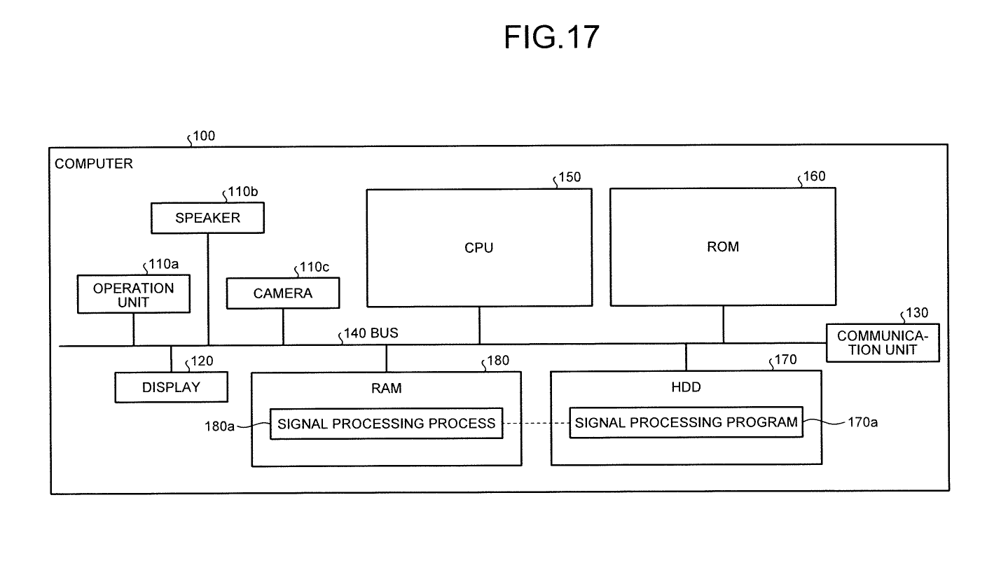

Various pieces of processing described in the above embodiments can be implemented when a computer such as a personal computer and a workstation executes a program prepared in advance. The following describes an example of a computer that executes a signal processing program having the same function as that in the embodiments described above with reference to FIG. 17.

FIG. 17 is a diagram for explaining an example of the computer that executes the signal processing program according to the first embodiment to the third embodiment. As illustrated in FIG. 17, a computer 100 includes an operation unit 110a, a speaker 110b, a camera 110c, a display 120, and a communication unit 130. The computer 100 further includes a CPU 150, a ROM 160, an HDD 170, and a RAM 180. The components 110 to 180 are connected to each other via a bus 140.

As illustrated in FIG. 17, a signal processing program 170a that exhibits the same function as that of the signal processing unit 17 described in the first embodiment is stored in the HDD 170 in advance. Similarly to the components of the signal processing unit 17 illustrated in FIG. 1, the signal processing program 170a may be appropriately integrated or separated. That is, all pieces of data are not necessarily stored in the HDD 170 at all times. Only pieces of data for processing may be stored in the HDD 170.

The CPU 150 then reads out the signal processing program 170a from the HDD 170, and loads the signal processing program 170a into the RAM 180. Accordingly, as illustrated in FIG. 17, the signal processing program 170a functions as a signal processing process 180a. The signal processing process 180a appropriately loads the various pieces of data read from the HDD 170 into a region allocated to itself on the RAM 180, and performs various pieces of processing based on the various pieces of loaded data. The signal processing process 180a includes processing performed by the signal processing unit 17 illustrated in FIG. 1, for example, the processing illustrated in FIG. 11 and FIG. 16. Regarding processing units to be virtually implemented on the CPU 150, all the processing units do not necessarily operate on the CPU 150 at all times. Only the processing units for processing may be virtually implemented.

The signal processing program 170a is not necessarily stored in the HDD 170 or the ROM 160 in advance. For example, each program is stored in a “portable physical medium” such as a flexible disk, what is called an FD, a CD-ROM, a DVD disc, a magneto-optical disc, and an IC card to be inserted into the computer 100. The computer 100 may acquire each program from the portable physical medium to execute the program. Each program may be stored in another computer or a server device connected to the computer 100 via a public network, the Internet, a LAN, a WAN, and the like so that the computer 100 acquires the program therefrom to execute the program.

Output control can be appropriately performed on the detection result of the pulse wave.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A signal processing device comprising:
a processor that executes a process comprising;
acquiring an image;
extracting a living body region included in the image;
first generating a signal from time series data of a pixel value included in a partial image of the image corresponding to the living body region;
calculating a variation index for evaluating a degree of disturbance of a pulse wave included in the signal; and
controlling whether to output the signal by using the variation index.

2. The signal processing device according to claim 1, wherein the calculating includes calculating an index related to a frequency region of the signal as the variation index.

3. The signal processing device according to claim 2, wherein the calculating includes calculating a ratio between a first peak and a second peak as the variation index, the first peak being highest and the second peak being second highest among peaks included in a spectrum of the signal.

4. The signal processing device according to claim 2, wherein the calculating includes calculating a size of an area of spectral distribution of the signal as the variation index.

5. The signal processing device according to claim 1, wherein the calculating includes calculating an index related to a time region of the signal as the variation index.

6. The signal processing device according to claim 5, wherein the calculating includes calculating, as the variation index, a standard deviation of time intervals between intersection points of a waveform of the signal and a plurality of straight lines parallel with a time axis.

7. The signal processing device according to claim 5, wherein the calculating includes calculating, as the variation index, a standard deviation of a difference in amplitude between adjacent extreme values in a waveform of the signal.

8. The signal processing device according to claim 5, wherein the calculating includes calculating correlation coefficients between a waveform of the signal and a duplicated waveform obtained by duplicating part of the signal in a predetermined time width while shifting the duplicated waveform, and calculating a maximum value of the calculated correlation coefficients as the variation index.

9. The signal processing device according to claim 1, the process further comprising:
second generating, by using an error between the signal and an electrocardiographic signal corresponding to the signal, a determination model including a classification tree of a variation index and a threshold used for determination in the classification tree with which the highest percentage of correct answers are obtainable when the signal is classified into classes of good and poor based on a plurality of variation indices calculated at the calculating, wherein
the controlling includes controlling whether to output the signal in accordance with the determination model generated at the second generating by using the variation index calculated at the calculating.

10. The signal processing device according to claim 1, wherein
the calculating includes calculating a plurality of variation indices, and
the controlling includes giving a predetermined weight to the variation indices to synthesize the variation indices, and controlling whether to output the signal by comparing the synthesized variation index with a predetermined threshold.

11. The signal processing device according to claim 1, wherein the calculating includes calculating the variation index by using a sensor value obtained by a predetermined sensor.

12. A signal processing method comprising:
acquiring, by a processor, an image;
extracting, by the processor, a living body region included in the image;
generating, by the processor, a signal from time series data of a pixel value included in a partial image of the image corresponding to the living body region;
calculating, by the processor, a variation index for evaluating a degree of disturbance of a pulse wave included in the signal; and
controlling, by the processor, whether to output the signal by using the variation index.

13. A non-transitory computer-readable recording medium having stored therein a signal processing program that causes a computer to execute a process comprising:
acquiring an image;
extracting a living body region included in the image;
generating a signal from time series data of a pixel value included in a partial image of the image corresponding to the living body region;
calculating a variation index for evaluating a degree of disturbance of a pulse wave included in the signal; and
controlling whether to output the signal by using the variation index.
